(19) United States
(12) Patent Application Publication
(10) Pub. No.: US 2025/0346545 A1
(43) Pub. Date: Nov. 13, 2025

(54) COMBINING OXIDATIVE COUPLING OF METHANE WITH ADIABATIC THERMAL CRACKING (PYROLYSIS) REACTOR

(71) Applicant: SABIC GLOBAL TECHNOLOGIES B.V., BERGEN OP ZOOM (NL)

(72) Inventors: Byeongjin BAEK, Bangalore (IN); Murali GOPALAKRISHNAN, Bangalore (IN); Gaurav ARORA, Bangalore (IN); Atul PANT, Bangalore (IN)

(73) Assignee: SABIC GLOBAL TECHNOLOGIES B.V., BERGEN OP ZOOM (NL)

(21) Appl. No.: 18/860,251

(22) PCT Filed: May 12, 2023
(86) PCT No.: PCT/EP2023/062808

§ 371 Date: Oct. 25, 2024

(30) Foreign Application Priority Data
May 13, 2022 (EP) 22173245.6
Aug. 2, 2022 (EP) 22188285.5

(51) Int. Cl. C07C 2/82; B01J 6/00; B01J 19/00; B01J 19/24

(52) U.S. Cl. CPC C07C 2/82; B01J 6/008; B01J 19/0006; B01J 19/245; B01J 2219/00186

(57) ABSTRACT

A method of producing olefins by producing an oxidative coupling of methane (OCM) product comprising olefins via oxidative coupling of methane, subjecting an adiabatic thermal cracking (ATC) feed stream comprising at least a portion of the OCM product to ATC (or “pyrolysis”) to produce an ATC product, and controlling a mole ratio of oxygen to ethane in the ATC feed stream within a range of greater than zero and less than 0.25.

### CROSS REFERENCE TO RELATED APPLICATIONS

N/A.

### TECHNICAL FIELD

The present disclosure relates to methods of producing hydrocarbons, more specifically methods of producing olefins, such as ethylene and propylene by oxidative coupling of methane integrated with adiabatic thermal cracking.

### BACKGROUND

Hydrocarbons, and specifically olefins such as ethylene (C₂H₄) and propylene (C₃H₆), are typically building blocks used to produce a wide range of products, for example, break-resistant containers and packaging materials. Currently, for industrial scale applications, olefins are produced by heating natural gas condensates and petroleum distillates, which include ethane and higher hydrocarbons, and the produced ethylene is separated from a product mixture by using gas separation processes.

Oxidative coupling of the methane (OCM) has been the target of intense scientific and commercial interest for more than thirty years due to the tremendous potential of such technology to reduce costs, energy, and environmental emissions in the production of C₂H₄. As an overall reaction, in the OCM, CH₄ and O₂ react exothermically over a catalyst to produce hydrocarbons, including C₂H₄, water (H₂O) and heat.

Thus, there is an ongoing need for the development of OCM processes that can increase the production of olefins.

### BRIEF SUMMARY

Disclosed herein is a method comprising: producing an oxidative coupling of methane (OCM) product comprising olefins via oxidative coupling of methane; subjecting an adiabatic thermal cracking (ATC) feed stream comprising at least a portion of the OCM product to ATC (also referred to as “pyrolysis”) to produce an ATC product; and controlling a mole ratio of oxygen to ethane in the ATC feed stream within a range of greater than zero and less than 0.25 (e.g., 0.005 to 0.25, 0.01 to 0.25, 0.05 to 0.25, 0.005 to 0.2, 0.01 to 0.2, 0.05 to 0.2).

Also disclosed herein is a system comprising: an oxidative coupling of methane (OCM) reaction zone configured to produce an OCM product comprising olefins via oxidative coupling of methane; and an adiabatic thermal cracking (ATC) reaction zone configured to produce an ATC product by subjecting a feed stream comprising at least a portion of the OCM product to ATC, wherein a mole ratio of oxygen to ethane in the ATC feed stream is within a range of greater than zero and less than 0.25 (e.g., 0.005 to 0.25, 0.01 to 0.25, 0.05 to 0.25, 0.005 to 0.2, 0.01 to 0.2, 0.05 to 0.2).

Further disclosed herein is a method comprising: producing an oxidative coupling of methane (OCM) product comprising olefins via oxidative coupling of methane; and subjecting an adiabatic thermal cracking (ATC) feed stream comprising at least a portion of the OCM product to ATC to produce an ATC product, wherein a residence time (tᵣₑₛ) of the subjecting of the ATC feed stream to ATC is in a range of from about 200 to about 1000 milliseconds (ms), from about 200 to about 800 ms, or from about 450 to about 550 ms, or less than or equal to about 1000, 900, 800, 700, 600, 550, 500, 450, 400, 350, 300, 250, or 200 milliseconds (ms).

### BRIEF DESCRIPTION OF THE DRAWINGS

For a detailed description of embodiments of the disclosed methods, reference will now be made to the accompanying drawing in which:

FIG. 1 is a schematic of a method, according to embodiments of this disclosure;

FIG. 2 is a schematic of a system, according to embodiments of this disclosure;

FIG. 3 is a schematic of an example integrated reactor, according to embodiments of this disclosure;

FIG. 4 is a schematic of an example separation apparatus, according to embodiments of this disclosure;

FIG. 5 is a schematic of temperature as a function of residence time (tᵣₑₛ) in ATC reaction zone/reactor of the simulation described in the Example;

FIG. 6 is a schematic of ethane mass fraction as a function of residence time (tᵣₑₛ) in ATC reaction zone/reactor of the simulation described in the Example;

FIG. 7 is a schematic of olefin mass fraction as a function of (tᵣₑₛ) in ATC reaction zone/reactor of the simulation described in the Example; and

FIG. 8 is a schematic of C4+ mass fraction as a function of (tᵣₑₛ) in ATC reaction zone/reactor of the simulation described in the Example.

### DETAILED DESCRIPTION

Disclosed herein are a system and method that employ a novel design and operation of an adiabatic thermal cracking (ATC) reactor or reaction zone (also referred to herein as a, “pyrolysis reactor or reaction zone” or a “post-catalytic reactor or reaction zone”) that is combined with an oxidative coupling of methane (OCM) reactor or reaction zone to maximize the production of olefins (e.g., ethylene, propylene). The effluent of the oxidative methane coupling reactor or reaction zone contains paraffins (e.g., ethane, propane, and butane) that can be thermally cracked in the downstream pyrolysis reactor utilizing the high temperature generated by OCM reactions in the OCM reactor or reaction zone. The design of the pyrolysis reactor or reaction zone is different from existing steam cracking furnaces. In the pyrolysis reactor or zone of this disclosure, conversion of ethane can range from 50% to 70% depending on the effluent or “OCM product” of the OCM reactor. The OCM reactor is operated under conditions to achieve an effluent temperature that is high enough to drive the reaction to the ethane conversion level of 50 to 70%; hence, via the system and method of this disclosure, there is no need to supply heat to the pyrolysis reactor. In general, thermal cracking of hydrocarbons involves the formation of heavies and coke (soot); thus, the system and method of this disclosure provide an optimal residence time (tᵣₑₛ) in the ATC reactor to minimize loss of hydrocarbons to heavies. In embodiments, a residence time of around 200 to 1000 milliseconds can provide ethane conversion of greater than 50%, with a minimum loss of carbon to heavies (e.g., benzene, MAPD, butadiene, cyclopentadiene, etc.)

In embodiments, a process for producing ethylene is provided that combines OCM and novel ATC, where ethane in an OCM product or effluent of the OCM is converted to olefins, including ethylene and propylene, utilizing the high temperature generated by OCM reactions and oxygen slip from the OCM reactor or reaction zone to the ATC, where an optimal residence time in the integrated ATC can be about 500 to about 1000 milliseconds, and can maximize the olefin production while minimizing a loss of carbon to heavies (e.g., C4+). As detailed hereinbelow, oxygen slip can be optimized to a level where the mild oxidation with the oxygen slip provides extra heat for endothermic ATC reactions. In embodiments, the oxygen slip (e.g., mole ratio of O₂/ethane) can be maintained less than 0.25 or 0.2 (molar basis).

As utilized herein, “C2+” indicates hydrocarbons (e.g., alkanes, alkenes, alkynes), having two or more carbon atoms, “C3+” indicates hydrocarbons (e.g., alkanes, alkenes, alkynes) having three or more carbons, and “C4+” indicates hydrocarbons (e.g., alkanes, alkenes, alkynes) having four or more carbon atoms. Similarly, “C2+ alkanes” indicate alkanes having two or more carbon atoms, “C3+ alkanes” indicate alkanes having three or more carbon atoms, and “C4+ alkanes” include alkanes having four or more carbon atoms, while “C2+ olefins” indicate olefins having two or more carbon atoms, “C3+ olefins” indicate olefins having three or more carbon atoms, and “C4+ olefins” include olefins having four or more carbon atoms, and “C2+ alkynes” indicate alkynes having two or more carbon atoms, “C3+ alkynes” indicate alkynes having three or more carbon atoms, and “C4+ alkynes” include alkynes having four or more carbon atoms.

Other than in the Example or where otherwise indicated, all numbers or expressions referring to quantities of ingredients, reaction conditions, and the like, used in the specification and claims are to be understood as modified in all instances by the term “about.” Various numerical ranges are disclosed herein. Because these ranges are continuous, they include every value between the minimum and maximum values. The endpoints of all ranges reciting the same characteristic or component are independently combinable and inclusive of the recited endpoint. Unless expressly indicated otherwise, the various numerical ranges specified in this application are approximations. The endpoints of all ranges directed to the same component or property are inclusive of the endpoint and independently combinable. The term “from more than 0 to an amount” means that the named component is present in some amount more than 0, and up to and including the higher named amount.

The terms “a,” “an,” and “the” do not denote a limitation of quantity, but rather denote the presence of at least one of the referenced item. As used herein the singular forms “a,” “an,” and “the” include plural referents.

As used herein, “combinations thereof” is inclusive of one or more of the recited elements, optionally together with a like element not recited, e.g., inclusive of a combination of one or more of the named components, optionally with one or more other components not specifically named that have essentially the same function. As used herein, the term “combination” is inclusive of blends, mixtures, alloys, reaction products, and the like.

Reference throughout the specification to “an aspect,” “another aspect,” “other aspects,” “some aspects,” and so forth, means that a particular element (e.g., feature, structure, property, and/or characteristic) described in connection with the aspect is included in at least an aspect described herein, and may or may not be present in other aspects. In addition, it is to be understood that the described element(s) can be combined in any suitable manner in the various aspects.

As used herein, the terms “inhibiting” or “reducing” or “preventing” or “avoiding” or any variation of these terms, include any measurable decrease or complete inhibition to achieve a desired result.

As used herein, the term “effective,” means adequate to accomplish a desired, expected, or intended result.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”) are inclusive or open-ended and do not exclude additional, unrecited elements or method steps.

Unless defined otherwise, technical and scientific terms used herein have the same meaning as is commonly understood by one of skill in the art.

Compounds are described herein using standard nomenclature. For example, any position not substituted by any indicated group is understood to have its valency filled by a bond as indicated, or a hydrogen atom.

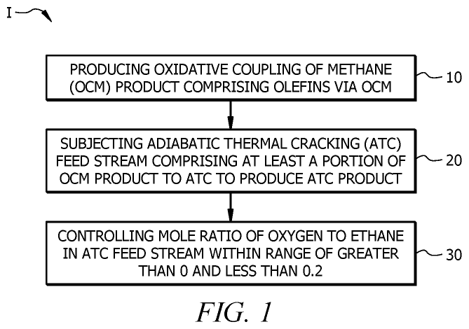

FIG. 1 is a schematic of a method I, according to embodiments of this disclosure. Method I comprises: at 10, producing an oxidative coupling of methane (OCM) product comprising olefins via oxidative coupling of methane; at 20, subjecting an adiabatic thermal cracking (ATC) feed stream comprising at least a portion of the OCM product to ATC (or “pyrolysis”) to produce an ATC product; and, at 30, controlling a mole ratio of oxygen to ethane in the ATC feed stream within a range of greater than zero and less than 0.25. The oxygen in the ATC feed stream (at least a portion of which can be provided by slipping through form the OCM reactions is sometimes referred to herein as oxygen “slip”.

Producing the OCM product comprising olefins via OCM, at 10, can be effected via any suitable systems and methods. By way of non-limiting examples, in embodiments, the OCM product can be produced substantially as described in U.S. Pat. Nos. 10,329,215, 10,843,982, 10,941,088, 11,148,985, or U.S. Patent Application 2021/0031161 the disclosure of each of which is incorporated herein in its entirety for purposes not contrary to this disclosure.

Ethylene can be selectively produced by OCM as represented by Equations (I):

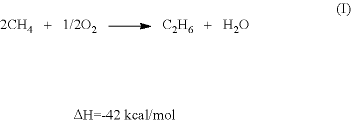

Overall, the preferred oxidative coupling of methane to ethane is exothermic. Side reactions in OCM represented by Equation (II) and (III) are also exothermic:

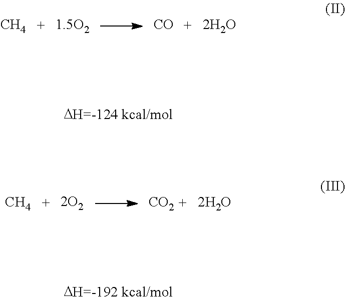

The excess heat from the reactions in Equations (II) and (III) further increases reactor temperature, thereby, potentially leading to thermal runaway and substantially reducing the selectivity of C₂ production.

Methane is a chemically stable molecule owing to the presence of its four strong tetrahedral C—H bonds (435 kJ/mol). When catalysts are used in the OCM, the energy barrier to break the C—H bond in methane can be significantly reduced, which in turn decreases the rates of unwanted side reactions and increases the ethylene selectivity.

Generally, in the OCM, CH₄ can be oxidatively converted into ethane (C₂H₆), and partial dehydrogenation of C₂H₆ can produce C₂H₄ and C₂H₂. CH₄ is activated heterogeneously on a catalyst surface, forming methyl free radicals (e.g., CH₃—), which then couple in a gas phase to form C₂H₆. C₂H₆ can subsequently undergo dehydrogenation to form C₂H₄ and C₂H₂. An overall yield of desired C₂ hydrocarbons is reduced by non-selective reactions of methyl radicals with oxygen on the catalyst surface and/or in the gas phase, which produce (undesirable) carbon monoxide and carbon dioxide. Some of the best reported OCM outcomes encompass about a 20% conversion of methane and about an 80% selectivity to desired C₂ hydrocarbons.

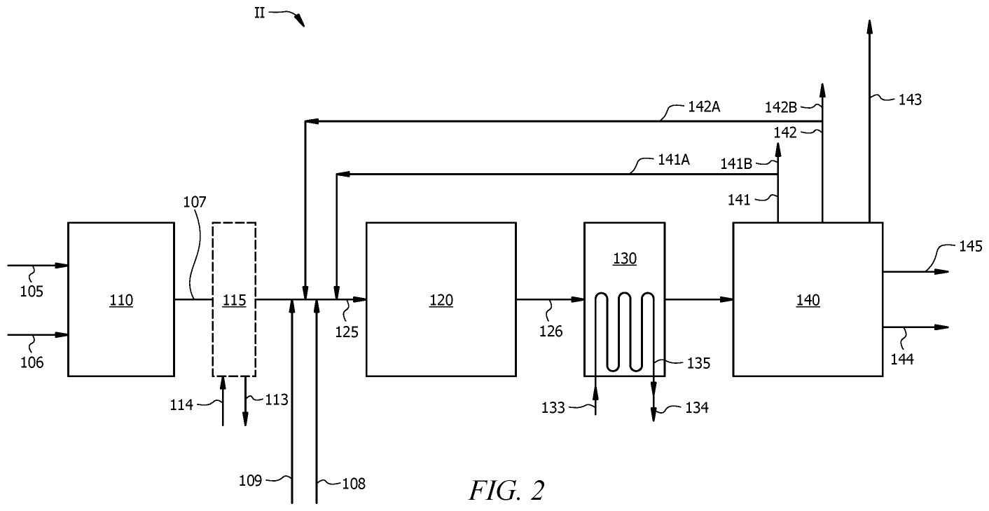

The process of this disclosure will now be described with reference to FIG. 2, which is a schematic of a system II, according to embodiments of this disclosure. As will be appreciated by one of skill in the art, and with the help of this disclosure, system components shown in FIG. 2 can be in fluid communication with each other (as represented by the connecting lines indicating a direction of fluid flow) through any suitable conduits (e.g., pipes, streams, etc.).

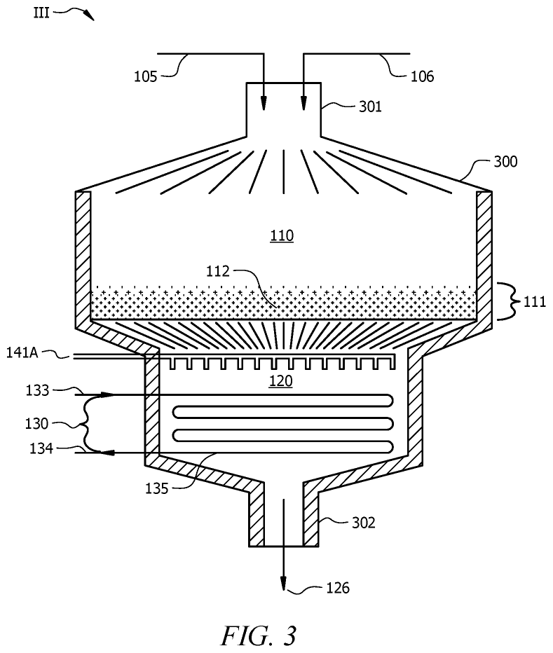

As seen in FIG. 2, the producing of the OCM product can be effected in an OCM reactor or reaction zone 110 (hereinafter referred to as “OCM reaction zone 110”). Methane (CH₄) 105 (e.g., a stream or gas comprising methane) and oxygen (O₂) 106 (e.g., a stream or gas comprising oxygen) can be introduced into OCM reactor or reaction zone 110, and OCM product 107 removed therefrom. In embodiments, a process for producing ethylene and syngas as disclosed herein can comprise reacting, via an oxidative coupling of methane (OCM) reaction, an OCM reactant mixture in the OCM reaction zone 110 to produce OCM product 107, wherein the OCM reaction zone 110 comprises an OCM catalyst 112 (FIG. 3, hereinbelow), wherein the OCM reactant mixture comprises methane (CH₄) containing stream 105 and oxygen (O₂) stream 106, and wherein the OCM product mixture 107 comprises ethylene (C₂H₄), ethane (C₂H₆), hydrogen (H₂), carbon monoxide (CO), carbon dioxide (CO₂), and unreacted methane, and other C2+ hydrocarbons.

In embodiments, the OCM reaction zone 110 (e.g., OCM reactor, common reactor) can comprise an adiabatic reactor, an autothermal reactor, a tubular reactor, a continuous flow reactor, and the like, or combinations thereof. In embodiments, the OCM reaction zone 110 can be operated autothermally, providing sufficient cooling for removal of the large heat generated in the OCM reactions and/or heat can otherwise be removed, enabling high C2 selectivity (e.g., selectivity to ethylene, ethane, acetylene) and methane conversion (e.g., greater than about 15%, 15%-20%) within a single OCM reactor or zone 110.

The effluent of the OCM reaction zone 110 can vary, depending on the oxygen provided by oxygen containing stream 105 and methane provided by methane containing stream 106 conversions in the OCM reaction zone 110. At a methane 105 conversion around 15 to 20%, ethane (C₂H₆) and propane (C₃H₈) concentrations can range from 2 to 8%. The OCM reaction zone can have an operating temperature of 880° C.±20° C., for example, in a range of from about 860 to 900° C., from about 870 to about 890° C., or from about 875 to about 885° C. An operating pressure in the OCM reaction zone 110 can be in a range of from about 2 to about 10 bar (0.2 MPa to 1.0 MPa), from about 3 to about 10 bar (0.3 MPa to 1.0 MPa), from about 4 to about 10 bar (0.4 MPa to 1.0 MPa), or from about 5 to about 10 bar (0.5 MPa to 1.0 MPa). In embodiments, the operating pressure in the OCM reaction zone 110 is greater than or equal to about 1, 1.5, 2, 2.5, 3, 3.5, 4, 4.5, 5, 5.5, 6, 6.5, 7, 7.5, 8, 8.5, 9, 9.5, or 10 bar (0.1, 0.15, 0.2, 0.25, 0.3, 0.35, 0.4, 0.45, 0.5, 0.55, 0.6, 0.65, 0.7, 0.75, 0.8, 0.85, 0.9, 0.95, or 1.0 MPa).

Producing the OCM product 107 comprising olefins via OCM at 10 can be effected in the presence of an OCM catalyst. The OCM catalyst can comprise one or more oxides, such as basic oxides; mixtures of basic oxides; redox elements; redox elements with basic properties; mixtures of redox elements with basic properties; mixtures of redox elements with basic properties promoted with alkali and/or alkaline earth metals; rare earth metal oxides; mixtures of rare earth metal oxides; mixtures of rare earth metal oxides promoted by alkali and/or alkaline earth metals; manganese; manganese compounds; lanthanum; lanthanum compounds; sodium; sodium compounds; cesium; cesium compounds; calcium; calcium compounds; and the like; or combinations thereof.

In embodiments, the OCM catalysts suitable for use in the present disclosure can be supported catalysts and/or unsupported catalysts. In embodiments, the supported catalysts can comprise a support, wherein the support can be catalytically active (e.g., the support can catalyze an OCM reaction). For example, the catalytically active support can comprise a metal oxide support, such as MgO. In other embodiments, the supported catalysts can comprise a support, wherein the support can be catalytically inactive (e.g., the support cannot catalyze an OCM reaction), such as SiO₂. In yet other embodiments, the supported catalysts can comprise a catalytically active support and a catalytically inactive support.

In embodiments, the support comprises an inorganic oxide, alpha, beta or theta alumina (Al₂O₃), activated Al₂O₃, silicon dioxide (SiO₂), titanium dioxide (TiO₂), magnesium oxide (MgO), calcium oxide (CaO), strontium oxide (SrO), zirconium oxide (ZrO₂), zinc oxide (ZnO), lithium aluminum oxide (LiAlO₂), magnesium aluminum oxide (MgAlO₄), manganese oxides (MnO, MnO₂, Mn₃O₄), lanthanum oxide (La₂O₃), activated carbon, silica gel, zeolites, activated clays, silicon carbide (SiC), diatomaceous earth, magnesia, aluminosilicates, calcium aluminate, carbonates, MgCO₃, CaCO₃, SrCO₃, BaCO₃, Y₂(CO₃)₃, La₂(CO₃)₃, and the like, or combinations thereof. In embodiments, the support can comprise MgO, Al₂O₃, SiO₂, ZrO₂, and the like, or combinations thereof.

Nonlimiting examples of OCM catalysts suitable for use in the present disclosure include CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, Sr/Mn—Na₂WO₄, and the like, or combinations thereof.

The OCM reactant mixture can comprise a hydrocarbon or mixtures of hydrocarbons, provided by methane containing stream 105 and oxygen in oxygen containing stream 106. That is, methane stream 105 can comprise hydrocarbons or mixtures of hydrocarbons including natural gas (e.g., CH₄), liquefied petroleum gas comprising C₂-C₅ hydrocarbons, C₆₊ heavy hydrocarbons (e.g., C₆ to C₂₄ hydrocarbons, such as diesel fuel, jet fuel, gasoline, tars, kerosene, etc.), oxygenated hydrocarbons, biodiesel, alcohols, dimethyl ether, and the like, or combinations thereof. In embodiments, the OCM reactant mixture can comprise CH₄ provided as or by methane containing stream 105 and O₂ provided as or by oxygen containing stream 106.

Oxygen containing stream 106 can be or comprise oxygen gas (which may be obtained via a membrane separation process), technical oxygen (which may contain some air), air, oxygen enriched air, and the like, or combinations thereof.

The OCM reactant mixture can further comprise a diluent that can be introduced separately to OCM reaction zone 110, or in combination with methane containing stream 105 or oxygen containing stream 106. The diluent can be inert with respect to the OCM reaction, e.g., the diluent may not participate in the OCM reaction. In embodiments, the diluent can comprise water, steam, nitrogen, inert gases (e.g., argon), and the like, or combinations thereof. In embodiments, the diluent can be present in the reactant mixture in an amount of from about 0.5% to about 80%, alternatively from about 5% to about 50%, or alternatively from about 10% to about 30%, based on the total volume of the OCM reactant mixture.

In embodiments, the OCM reactant mixture can be characterized by a CH₄/O₂ molar ratio of from about 2:1 to about 10:1, alternatively from about 3:1 to about 9:1, or alternatively from about 4:1 to about 8:1.

The OCM product 107 can comprise ethane in an amount of from about 1 mol % to about 20 mol %, alternatively from about 2.5 mol % to about 15 mol %, alternatively from about 5 mol % to about 10 mol %, or alternatively from about 5 mol % to about 7.5 mol %.

The OCM product 107 can comprise CO₂ in an amount of from about 1 mol % to about 20 mol %, alternatively from about 5 mol % to about 15 mol %, alternatively from about 7 mol % to about 13 mol %, or alternatively from about 8 mol % to about 12 mol %.

As noted above, a method of this disclosure comprises, at 20 of FIG. 1, subjecting an ATC feed stream comprising at least a portion of the OCM product to ATC to produce an ATC product. As depicted in FIG. 2, at least a portion of the OCM product 107 can be introduced into ATC reactor or ATC reaction zone 120 (hereinafter referred to as, “ATC reaction zone 120” or “post catalytic reaction zone”), within which adiabatic thermal cracking of the ATC feed stream produces ATC product 126.

Subjecting the ATC feed stream 125 to ATC to produce the ATC product 126 can be effected at a pressure substantially equal to an operating pressure of OCM reaction zone 110. For example, in embodiments, subjecting the ATC feed stream 125 to ATC to produce the ATC product 126 can be effected at an ATC operating pressure of greater than or equal to about 1, 2, 3, 4, 5, 6, 7, 8, 9, or 10 bars (0.1 MPa, 0.2 MPa, 0.3 MPa, 0.4 MPa, 0.5 MPa, 0.6 MPa, 0.7 MPa, 0.8 MPa, 0.9 MPa, or 1.0 MPa). For example, the ATC operating pressure can be in a range of from about 4 to about 6 bars (from about 0.4 MPa to about 0.6 MPa), from about 4.5 to about 10 bars (from about 0.45 MPa to about 1.0 MPa), or from about 5 to about 10 bars (from about 0.5 MPa to about 1.0 MPa).

In embodiments, a residence time (tᵣₑₛ), during the subjecting of the ATC feed stream to ATC, can be in a range of from about 200 to about 1000 milliseconds (ms), from about 200 to about 800 ms, from about 450 to about 550 ms, less than or equal to about 1000, 900, 800, 700, 600, 550, 500, 450, 400, 350, 300, 250, or 200 milliseconds (ms), and/or greater than or equal to about 300, 350, or 400 ms.

The method can further comprise quenching the ATC product 125 immediately after the residence time (tᵣₑₛ). Quenching can comprise indirectly contacting the ATC product 125 with a heat transfer fluid. For example, quenching can be effected in a quenching zone 130 immediately downstream of the ATC reaction zone 120, as depicted in FIG. 2. Quenching zone 130 can comprise a heat exchanger 135 configured to reduce a temperature of the ATC product 125 via indirect contact thereof with a heat transfer fluid 133, thus forming a heated heat transfer fluid 135 having a temperature greater than a temperature of the heat transfer fluid 133 introduced into heat exchanger 135. In embodiments, the heat transfer fluid comprises water or steam and the heated heat transfer fluid comprises steam. The quenching can be effected to reduce a temperature of the ATC product 125 to a quenched temperature. The quenched temperature can be a temperature below which most undesirable olefin cracking reactions are ceased, for example, a quenched temperature of less than or equal to about 600, 550, or 500° C.

In embodiments, a C4+ mass fraction in the ATC product 126 is less than or equal to about 0.004, 0.003, or 0.002.

In embodiments, the ATC is effected without heating the ATC feed stream 125 (e.g., without utilizing a furnace or other heater to increase a temperature of the ATC feed stream 125).

As indicated at 30 of FIG. 1, a method of this disclosure can further comprise controlling a mole ratio of oxygen (O₂) to ethane (C₂H₆) in the ATC feed stream 125. For example, the method can comprise controlling the mole ratio of oxygen to ethane in the ATC feed stream 125 to less than or equal to about 0.25, 0.24, 0.23, 0.22, 0.21, 0.20, 0.19, 0.18, 0.17, 0.16, 0.15, 0.14, 0.13, 0.12, 0.11, 0.1, 0.9, 0.8, 0.7, 0.6, 0.5, 0.4, 0.3, 0.2, 0.1, or 0.001. For example, the mole ratio of oxygen to ethane can be in a range of from about 0.005 to about 0.2, from about 0.01 to about 0.2, or from about 0.05 to about 0.2, from about 0.005 to about 0.25, from about 0.01 to about 0.25, or from about 0.05 to about 0.25.

Controlling the mole ratio of oxygen to ethane in the ATC feed stream 125 can comprise adjusting a mole ratio of oxygen to ethane in the at least the portion of the OCM product 107 in the ATC feed stream 125, adjusting an amount of ethane and/or oxygen that is introduced into the ATC feed stream 125 separately from the at least the portion of the OCM product 107, or a combination thereof. Adjusting the mole ratio of the oxygen to ethane in the at least the portion of the OCM product 125 in the ATC feed stream can comprise changing the operation of OCM reaction zone 110 to alter the mole ratio of oxygen to ethane in the OCM product and/or separating oxygen from or adding oxygen to the at least the portion of the OCM product 107 utilized in the ATC feed stream 125. The OCM reaction zone 110 can be operated to adjust a molar ratio of oxygen to ethane in the OCM product 107. For example, altering a temperature, pressure, catalyst amount, feed preheat, feed flow, and/or cross sectional area of the OCM during the producing of the OCM product 107 in OCM reaction zone 110 can be utilized to alter the mole ratio of the oxygen to ethane in the OCM product 107. Alternatively or additionally, an amount of methane containing stream 105 and/or oxygen containing stream 106 utilized during the producing of the OCM product 107 can be employed to adjust the mole ratio of oxygen to ethane in the OCM product 107.

As noted above, adjusting the mole ratio of the oxygen to ethane in the OCM product 107 can comprise separating oxygen from or adding oxygen to the at least the portion of the OCM product 107 utilized in the ATC feed stream 125. With reference to FIG. 2, system II can further comprise a separator or “operation unit” 115 configured to adjust the mole ratio of the oxygen to ethane in the OCM product 107 (or the at least the portion of the OCM product 107 utilized in ATC feed stream 125) by separating oxygen 113 from or adding oxygen 114 to the (e.g., at least the portion of) the OCM product 107 in the ATC feed stream 125. In embodiments, separator 115 can comprise a catalyst within or upstream of the ATC reaction zone 120.

As noted above, controlling the mole ratio of oxygen to ethane in the ATC feed stream 125 can comprise adjusting an amount of ethane and/or oxygen that is introduced into the ATC feed stream 125 separately from the at least the portion of the OCM product 107. Adjusting the amount of ethane and/or oxygen introduced into the ATC feed stream 125 separately from the at least the portion of the OCM product 107 can comprise separating ethane from the (e.g., quenched) ATC product 126 and adjusting an amount of ethane (e.g., “supplemental ethane”) separated from the ATC product 126 and combined with the at least the portion of the OCM product 107 to provide the ATC feed stream 125.

Accordingly, one or more lines (e.g., ethane recycle line 141A, described hereinbelow, C3+ alkanes recycle line 142A) to the ATC reaction zone 120 whereby an amount of ethane propane, and/or oxygen can be introduced into the ATC feed stream 125 separately from the at least the portion of the OCM product 107, whereby the mole ratio of oxygen to ethane and/or propane in the ATC feed stream 125 can be maintained.

The ATC reaction zone 120 can exclude a catalyst, in embodiments. As will be appreciated by one of skill in the art, and with the help of this disclosure, while there are catalytic processes for hydrocarbon cracking (e.g., ethane cracking) and CO₂ hydrogenation, the current disclosure does not need a catalyst for ethane cracking and CO₂ hydrogenation in the ATC reaction zone 120; the ethane cracking in the ATC reaction zone 120 as disclosed herein is thermal in contrast to catalyzed.

In embodiments, at least a portion of ethane in the ATC feed stream 125 can undergo a cracking reaction to produce ethylene in the ATC reaction zone 120. Generally, a cracking reaction refers to a reaction by which a saturated hydrocarbon or mixture of saturated hydrocarbons is broken down into smaller molecules and/or unsaturated molecules. In the case of ethane cracking, C₂H₆ is converted to C₂H₄ and H₂ according to reaction (1):

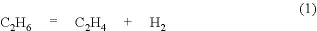

Cracking can be done in the presence of steam, and in this case it can be referred to as “steam cracking.” As will be appreciated by one of skill in the art, and with the help of this disclosure, steam for cracking can be supplied by the ATC feed stream 125 that contains at least a portion of the water from the OCM product 107. Further, as will be appreciated by one of skill in the art, and with the help of this disclosure, steam for cracking can be supplied by CO₂ hydrogenation reaction. As will be appreciated by one of skill in the art, and with the help of this disclosure, if water from the first product mixture and/or water produced by the CO₂ hydrogenation reaction is not sufficient for the needs of the steam cracking, additional steam can be introduced into the ATC reaction zone 120 as necessary. In embodiments, steam can be optionally introduced to the ATC reaction zone 120, for example via a dedicated steam feed line to the ATC reaction zone 120 and/or via addition to an existing feed stream (e.g., ethane recycle stream 141A) to the ATC reaction zone 120.

In embodiments, at least a portion of the carbon dioxide of the ATC feed stream 125 can undergo a hydrogenation reaction to carbon monoxide in the ATC reaction zone 120 according to reaction (2):

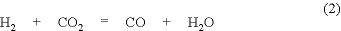

CO₂ hydrogenation can provide for an increased amount of CO in the ATC reaction zone 120, which overall can lead to a higher amount of syngas (H₂ and CO) in the ATC product 126. The amount of hydrogen present in the ATC reaction zone 120 will determine the H₂/CO molar ratio in the ATC product 126, as can be seen from reactions (3) and (4):

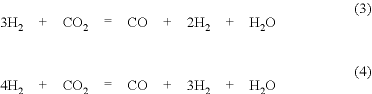

Reaction (2) is an equilibrium controlled reaction which depends on the H₂/CO₂ ratio, as it can be seen from reactions (3) and (4). Reactions (1) and (2) are endothermic, requiring relatively high temperatures, and as such reactions (1) and (2) can utilize the heat produced by the exothermic catalytic OCM reaction.

As noted above, in embodiments, the ATC feed stream 125 can comprise at least a portion of the OCM product 107 and supplemental ethane (e.g., in ethane recycle line 141A and/or fresh ethane line 108) and optionally supplemental CO₂ in fresh CO₂ line 109.

In embodiments, the supplemental ethane can comprise ethane recovered from the ATC product 126, as described herein. In other embodiments, the supplemental ethane can comprise ethane (e.g., fresh ethane in fresh ethane line 108) from a source other that the ethane recovered from the ATC product 126. In yet other embodiments, the supplemental ethane can comprise both (i) ethane recovered from the ATC product 126 (e.g., in ethane recycle line 141A); and (ii) fresh ethane (e.g., in fresh ethane line 108).

In embodiments, supplemental CO₂ can comprise CO₂ recovered from the ATC product, e.g., in pre-treatment apparatus 405. In other embodiments, the supplemental CO₂ can comprise CO₂ (e.g., fresh CO₂) from a source other that the CO₂ recovered from the ATC product 126. In yet other embodiments, the supplemental CO₂ can comprise both (i) CO₂ recovered from the ATC product 126; and (ii) fresh CO₂ (e.g., in fresh CO₂ line 109).

In embodiments, supplemental ethane and supplemental CO₂ can be introduced to the ATC reaction zone 126 via a common stream. In embodiments, supplemental ethane and supplemental CO₂ can be introduced to the ATC reaction zone 120 via separate (e.g., distinct, different) streams.

The method of this disclosure can further comprise separating C2+ alkanes (e.g., ethane, propane, butane, etc.) and/or C3+ olefins (propylene, butene, etc.) from the (e.g., quenched) ATC product 126. The separating can be effected by any suitable apparatus for effecting the desired separations. For example, separation apparatus 140, as depicted in FIG. 2, can be configured to separate ethane in ethane line 141, and propane in line 142, thus providing a C4 (e.g. butane, butadiene etc.) product in line 143, propylene product 144 and an ethylene product 145. All or a portion of the ethane in ethane line 141, and/or propane in propane 142, can be recycled to ATC reaction zone 120. For example, conversion to olefins in ATC reaction zone 120 can be increased, in embodiments, by utilizing at least a portion of the separated C2+ alkanes (e.g., in ethane line 141 and/or propane in line 142) separated from the ATC product in the ATC feed stream 126. In embodiments, the ethane conversion provided by in ATC reaction zone 120 (e.g., in the post-catalytic reaction zone) of the system and/or method of this disclosure is greater than or equal to about 40, 45, 50, 55, 60, 65, or 70 mole percent (mol %).

For purposes of the disclosure herein, the conversion of a reagent is a percent (%) conversion based on moles converted. For example, the ethane conversion can be calculated by using the following equation:

$${C}_{2} {H}_{6} conversion = \frac{{C}_{{C}_{2} {H}_{6}}^{in} - {C}_{{C}_{2} {H}_{6}}^{o u t}}{{C}_{{C}_{2} {H}_{6}}^{n}} \times 100 \%$$

wherein

$${C}_{{C}_{2} {H}_{6}}^{i n}$$

=number of moles of C₂H₆ that entered the post-catalytic zone, both from the catalytic zone effluent (e.g., from OCM product 107), and from any recycled or additional ethane introduced with the ATC feed stream 125; and wherein

$${C}_{{C}_{2} {H}_{6}}^{o u t}$$

=number of moles C₂H₆ that were recovered from the ATC reaction zone 120.

Separation apparatus 140 can be configured to separate ethane 141 from the ATC product 126, and a recycle line 141A can be configured to recycle and combine at least a portion of the separated ethane in ethane line 141 with the at least the portion of the OCM product 107 to provide the ATC feed stream 125. As noted above, the separation apparatus 140 can be further configured to separate C3+ alkanes and/or C3+ olefins from the ATC product. For example, propane line 142 can remove separated propane from separation apparatus 140, and a propane recycle line 142A can be configured to recycle and combine at least a portion of the separated propane in propane line 142 with the at least the portion of the OCM product 107 to provide the ATC feed stream 125; as ethane line 141 can remove separated ethane from separation apparatus 140, and an ethane line 141A can be configured to recycle and combine at least a portion of the separated ethane in ethane line 141 with the at least the portion of the OCM product 107 to provide the ATC feed stream 125; an propylene line 144 can remove separated propylene from separation apparatus 140, and an ethylene line 145 can remove separated ethylene from separation apparatus 140. In embodiments, none of the ethane, propane are recycled to ATC reaction zone 120. For example, propane can be desired product, in embodiments. At least a portion 141B of ethane in ethane line 141 may not be recycled; at least a portion 142B of propane in propane line 142 may not be recycled. Separation apparatus 140 can include any apparatus operable to provide the desired separations. For example, separation apparatus 140 can include one or more de-methanizers, configured to separate C1 hydrocarbons (e.g., methane) from a de-methanizer feed stream introduced thereto, one or more de-ethanizers configured to separate C2 hydrocarbons (e.g., ethane, ethylene) from a de-ethanizer feed stream introduced thereto, one or more de-propanizers configured to separate C3 hydrocarbons (e.g., propane, propylene) from a de-propanizer feed stream introduced thereto, or a combination thereof. One or more de-methanizer can be upstream of one or more de-ethanizer, which can be upstream of one or more de-propanizer, whereby a de-methanizer product (e.g., bottoms) stream comprising C2+ hydrocarbons can be introduced as de-ethanizer(s) feed stream, and a de-ethanizer product (e.g., bottoms) stream comprising C3+ hydrocarbons can be introduced as a feed stream to the one or more de-propanizers.

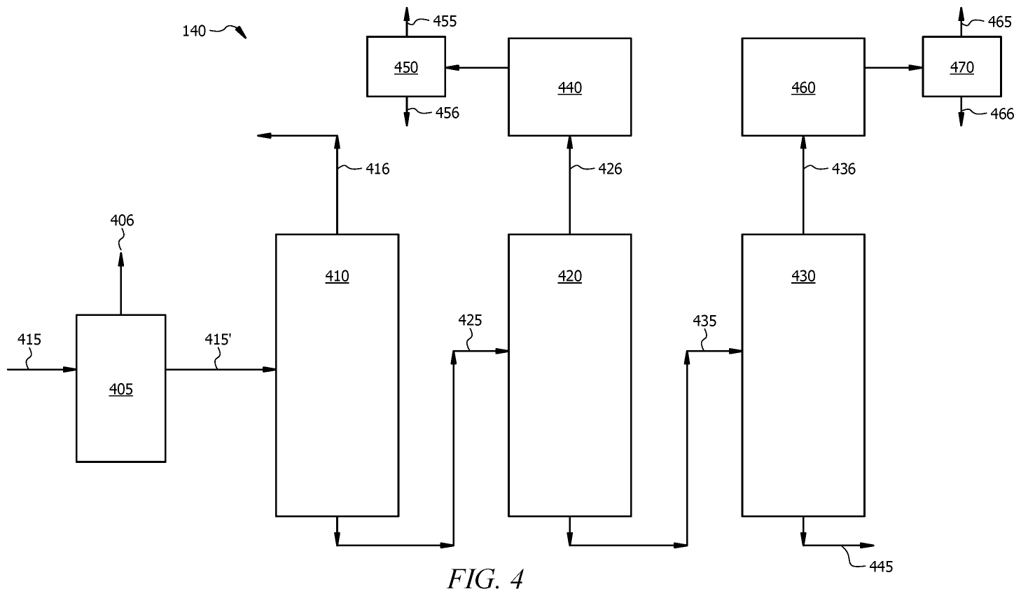

For example, as depicted in FIG. 4, which is a schematic of an example separation apparatus 140, de-methanizer 410 is configured to separate a de-methanizer feed stream 415 into a C1 stream 416 and a C2+ hydrocarbons stream, which can be utilized as de-ethanizer feed stream 425 to de-ethanizer 420. De-ethanizer 420 is configured to separate the de-ethanizer feed stream into a C2 stream 426 and a C3+ hydrocarbons stream, which can be utilized as de-propanizer feed stream 435 to de-propanizer 430. De-propanizer 430 is configured to separate the de-propanizer feed stream 435 into a C3 stream 436 and a C4+ stream 445.

In embodiments, the ATC product stream 126 (e.g., after quenching) can thus be introduced, before or after pre-treatment in a pre-treatment apparatus 405, into one or more de-methanizer column 410 as de-methanizer feed stream 415/415′. De-methanizer 410 is configured to produce a C1 product stream 416 comprising methane and a C2+ stream (e.g., a bottoms stream) comprising C2+ hydrocarbons from de-methanizer feed stream 415/pre-treated de-methanizer feed stream 415. The de-methanizer column 410 can be a cryogenic distillation column. The C1 product stream 416 can be recycled to OCM reaction zone 110 via methane feed stream 105. The C2+ hydrocarbons from de-methanizer 410 can be introduced to de-ethanizer column 420 as de-ethanizer feed stream 425. De-ethanizer 420 can separate the de-ethanizer feed stream 425 into a C2 hydrocarbons stream (e.g., overhead stream) and a C3+ hydrocarbons stream (e.g., bottoms stream). The de-ethanizer column 420 can be a cryogenic distillation column. The C2 hydrocarbons stream 426 can comprise ethylene (C₂H₄), ethane (C₂H₆), and acetylene (C₂H₂); and the C₃+ hydrocarbons stream 435 can comprise C3 hydrocarbons and C4 hydrocarbons.

In embodiments, at least a portion of the acetylene (C₂H₂) in the C2 hydrocarbons stream 426 can be contacted with H₂, for example in acetylene hydrogenation unit 440, to yield ethylene. Acetylene can be selectively hydrogenated to ethylene by using any suitable methodology, for example by gas phase hydrogenation.

In embodiments, an ethylene stream 455 and an ethane stream 456 can be recovered from at least a portion of the C2 hydrocarbons stream 426 by cryogenic distillation, for example in a C2 splitter column 450. All or a portion of ethane stream 456 can be recycled to the OCM reaction zone 110 and/or ATC reaction zone 120, for example, via ethane recycle line 141A, as described hereinabove.

In embodiments, a C3 hydrocarbons stream 436 and a C4+ hydrocarbons stream 445 can be recovered from at least a portion of the C3+ hydrocarbons stream 435, wherein the C3 hydrocarbons stream 436 comprise propylene (C₃H₆), and propane (C₃H₈). In embodiments, the C3+ hydrocarbons stream 435 can be conveyed from the de-ethanizer column 420 to a de-propanizer column 430 (e.g., a cryogenic distillation column) for the separation and recovery of C3 hydrocarbons 436. A methyl acetylene and propadiene (MAPD) hydrogenation 460 apparatus can be utilized to selectively hydrogenate methyl acetylene and propadiene in C3 hydrocarbons stream 460. In embodiments, a propylene stream 465 and an propane stream 466 can be recovered from at least a portion of the C3 hydrocarbons stream 436 by cryogenic distillation, for example in a C3 splitter column 470. All or a portion of propane stream 466 can be recycled to the OCM reaction zone 110 and/or ATC reaction zone 120, for example, via propane recycle stream 142A, as discussed hereinabove.

The separation apparatus 140 can further comprise pre-treatment apparatus 405 configured to remove one or more components (e.g., CO₂, water, sulfur) from the de-methanizer feed stream (e.g., from the quenched ATC product stream 126) to provide a pre-treated de-methanizer feed stream 415′.

In embodiments where the ethane recovered from the ATC product 126 is not sufficient for the amount of ethane necessary to be introduced to the ATC reaction zone 120 and/or the ethane recovered from the ATC product 126 cannot provide for the desired CO₂/ethane molar ratio (e.g., from about 0.8:1 to about 4.0:1) in the ATC feed stream 125, fresh ethane can be introduced to the ATC reaction zone 120 (e.g., via fresh ethane line 108).

In embodiments where the CO₂ recovered from the ATC product 126 is not sufficient for the amount of CO₂ to be introduced to the ATC reaction zone 120 and/or the CO₂ recovered from the ATC product 126 cannot provide for a desired CO₂/ethane molar ratio (e.g., from about 0.8:1 to about 4.0:1) in the ATC feed stream 125, fresh CO₂ can be introduced to the ATC reaction zone 120 (e.g., via fresh CO₂ line 109).

The ATC feed stream 125 can be characterized by an ethane and/or propane to methane molar ratio of from about 0.02:1 to about 0.04:1, alternatively from about 0.05:1 to about 0.06:1, alternatively from about 0.02:1 to about 0.07:1, or alternatively from about 0.03:1 to about 0.07:1.

The ATC feed stream 125 can be characterized by a CO₂ to methane molar ratio of from about 0.04:1 to about 0.06:1, alternatively from about 0.08:1 to about 0.1:1, alternatively from about 0.04:1 to about 0.2:1, or alternatively from about 0.1:1 to about 0.2:1.

In embodiments, the ATC reaction zone 120 can be characterized by a GHSV of from about 500 h⁻¹ to about 30,000 h⁻¹, alternatively from about 750 h⁻¹ to about 30,000 h⁻¹, alternatively from about 1,000 h⁻¹ to about 30,000 h⁻¹, alternatively from about 2,500 h⁻¹ to about 30,000 h⁻¹, alternatively from about 5,000 h⁻¹ to about 30,000 h⁻¹, alternatively from about 500 h⁻¹ to about 5,000 h⁻¹, alternatively from about 7,200 h⁻¹ to about 30,000 h⁻¹, alternatively from about 10,000 h⁻¹ to about 27,500 h⁻¹, or alternatively from about 12,500 h⁻¹ to about 25,000 h⁻¹.

Producing the OCM product 107 comprising olefins via OCM at 10 and the subjecting the ATC feed stream 125 comprising the at least a portion of the OCM product 107 to ATC to produce the ATC product 126 can be effected in a same vessel or in different vessels. The quenching of the ATC product 126 can be effected in a same vessel as the ATC, in embodiments.

FIG. 3 is a schematic of an integrated reactor III. Integrated reactor III comprises a vessel 300 containing OCM reaction zone 110, ATC reaction zone 120, and quenching zone 130. OCM reaction zone comprises a bed 111 of OCM catalyst 112. Methane containing stream 105 and oxygen containing stream 106 can be introduced into an inlet 301 of OCM reaction zone 110, which can be substantially frustoconical in shape. Downstream of OCM reaction zone 110 is ATC reaction zone 120, which can also be frustoconical in shape. Ethane recycle line 141A can be configured for introduction of ethane separated from ATC product 126 into ATC reaction zone 120, fresh ethane line 108 can be configured for introduction of fresh (e.g., non-recycled) ethane into ATC reaction zone 120, and/or CO₂ line 109 can be configured for introduction of CO₂ into ATC reaction zone 120. Quenching zone 130 comprising heat exchanger 135 can be positioned immediately downstream of or within ATC reaction zone 120. Quenched ATC product can be removed from integrated reactor III via outlet 302. Within quenching zone 130, heat exchange fluid 133 can be heated to provide heated heat exchange fluid (e.g., steam) 134. Other shapes and designs of integrated reactor III can be utilized to provide OCM reaction zone 110 upstream of ATC reaction zone 120 and/or quench zone 130 in a single integrated reactor III.

Also disclosed herein is a method comprising: producing an oxidative coupling of methane (OCM) product 107 comprising olefins via oxidative coupling of methane containing stream 105; and subjecting an adiabatic thermal cracking (ATC) feed stream 125 comprising at least a portion of the OCM product 107 to ATC (or “pyrolysis”) to produce an ATC product 126, wherein a residence time (tᵣₑₛ) of the subjecting of the ATC feed stream 125 to ATC is in a range of from about 200 to about 1000 milliseconds (ms), from about 200 to about 800 ms, or from about 450 to about 550 ms, or less than or equal to about 1000, 900, 800, 700, 600, 550, 500, 450, 400, 350, 300, 250, or 200 milliseconds (ms). The method can further comprise controlling a mole ratio of oxygen to ethane in the ATC feed stream 125 within a range of greater than zero and less than 0.25 (e.g., 0.005 to 0.25, 0.01 to 0.25, 0.05 to 0.25), as described hereinabove.

In embodiments, common reactor (e.g., integrated reactor III) comprises both the OCM reaction zone 110 and the ATC reaction zone 120. In embodiments, the integrated reactor III can comprise the OCM reaction zone 110 spanning across a first length of the reactor III, and the ATC reaction zone 120 spanning across a second length of the reactor III, wherein the first length plus the second length can sum up to a total length of the reactor III. As will be appreciated by one of skill in the art, and with the help of this disclosure, the OCM reaction zone 110 and the ATC reaction zone 120 in the common reactor III can be controlled by controlling the residence time in each of the reaction zones of a mixture traveling through the reactor III.

In embodiments, a boundary area between the OCM reaction zone 110 and the ATC reaction zone 120 can be a variable transitional boundary area (as opposed to a fixed, defined boundary, as it would be in the case when each of the OCM and the ATC reaction zones would be contained in different reactors, and not a common reactor III). Products from the OCM reaction zone 110 are communicated to the ATC reaction zone 120 via the OCM product 107. Depending on the type and configuration of the common reactor III used, the OCM product 107 may not be isolatable, owing to both the OCM reaction zone 110 and the ATC reaction zone 120 being contained in the common reactor III.

As noted hereinabove, the OCM reaction zone 110 can comprise a catalyst bed comprising the OCM catalyst. As will be appreciated by one of skill in the art, and with the help of this disclosure, the OCM reaction zone 110 can be considered to end where the catalyst bed ends, and the ATC reaction zone 120 can be considered to begin where the catalyst bed ends. The ATC reaction zone 120 can be proximate to the end of the catalyst bed contained in the OCM reaction zone 110. For purposes of the disclosure herein, the beginning and the end of a reaction zone (or catalyst bed contained in a reaction zone) are relative to the direction of flow through the system (e.g., through common reactor III).

The OCM reaction zone 110 can comprise OCM reaction zone inlet feed streams for the OCM reactant mixture, wherein the OCM reaction zone inlet feed streams can be located upstream of the OCM catalyst bed. The methane 105 and oxygen 106 can be introduced to the OCM reaction zone 110 upstream of the OCM catalyst bed. For purposes of the disclosure herein, the terms “upstream” and “downstream” are relative to the direction of flow through the system II (e.g., through common reactor III).

The ATC reaction zone 120 can comprise ATC reaction zone inlet feed streams for any supplemental ethane and/or supplemental CO₂, wherein the ATC reaction zone inlet feed streams can be located downstream (e.g., immediately downstream) of the OCM reaction zone 110, e.g., downstream or immediately downstream of the OCM catalyst bed. The ATC reaction zone inlet feed streams can be located downstream (e.g., immediately downstream) of the boundary area between the OCM reaction zone 110 and the ATC reaction zone 120, e.g., downstream or immediately downstream of the OCM catalyst bed.

In embodiments, the OCM reaction zone 110 and the ATC reaction zone 120 can be characterized by the same temperature and pressure.

In embodiments, the pressure in the OCM reaction zone 110 and the pressure in the ATC reaction zone 120 can be the same. In embodiments, the pressure in the OCM reaction zone 110 and the pressure in the ATC reaction zone 120 can be different.

In some embodiments, the temperature in the OCM reaction zone 110 and the temperature in the ATC reaction zone 120 can be the same. In other embodiments, the temperature in the OCM reaction zone 110 and the temperature in the ATC reaction zone 120 can be different. In embodiments, the ATC reaction zone 120 can be characterized by temperature of from about 860 to 900° C., from about 870 to about 890° C., or from about 875 to about 885° C. (e.g., 880° C.±20° C.).

In embodiments, a process for producing olefins as disclosed herein can comprise introducing at least a portion of the ATC product 126 to the separation apparatus 140, as described herein, for recovering a methane stream, an ethane stream, a CO₂ stream, an ethylene stream, a propylene stream, a propane stream, a C2+ alkanes stream (e.g., containing ethane, propane, and/or etc.), a C3+ alkanes stream (e.g., containing propane, butane, and/or etc.), a C4+ stream, or a combination thereof.

In embodiments, the separation apparatus 140 can employ distillation and/or cryogenic distillation to produce the methane stream, the ethylene stream, the ethane stream, the propane stream, and/the propylene stream; such distillation processes can occur subsequent to the recovery of a CO₂ stream from the ATC product 126.

In embodiments, the separation apparatus 140 can comprise any suitable separators, such as one or more distillation columns (e.g., cryogenic distillation columns); a water removal unit (e.g., a water quench vessel and/or a cooling tower); a CO₂ separator; etc.

In embodiments, the ATC product 126 further comprises water. In such embodiments, a process for producing olefins as disclosed herein can further comprise separating at least a portion of the water from the ATC product 126 prior to recovering the methane stream, the ethane stream, the carbon dioxide stream, the ethylene stream, the propane stream, the propylene stream, or the combination thereof from the at least a portion of the ATC product 126.

In embodiments, the ATC product 126 can be further compressed (e.g., via a compressor), for example to a pressure in the range of from about 350 psig to about 600 psig, alternatively about 400 psig to about 575 psig, or alternatively about 450 psig to about 500 psig, followed by optionally feeding the compressed second product mixture to a water removal unit. Generally, compressing a gas that contains water to increase its pressure will lead to the water condensing at the increased pressure at an increased temperature as compared to a temperature where water of an otherwise similar gas condenses at pressure lower than the increased pressure. The compressed ATC product 126 can be further introduced to a water removal unit (e.g., a water quench vessel and/or a cooling tower), where the compressed ATC product 126 can be further cooled to promote water condensation and removal.

In embodiments, a CO₂ stream can be recovered from the second product mixture by using a CO₂ separator of pre-treatment apparatus 405. The CO₂ separator can comprise CO₂ removal by amine (e.g., monoethanolamine) absorption (e.g., amine scrubbing), pressure swing adsorption, temperature swing adsorption, gas separation membranes (e.g., porous inorganic membranes, palladium membranes, polymeric membranes, zeolites, etc.), and the like, or combinations thereof. In embodiments, the CO₂ separator can comprise CO₂ removal by amine absorption.

In embodiments, at least a portion of the methane stream 416 can be recycled to the OCM reaction zone 110.

In embodiments, a process for olefins as disclosed herein can comprise recycling at least a portion of the ethane stream 456 to the ATC reaction zone 120. The recycled ethane stream in ethane recycle line 141A can be further contacted with additional ethane prior to recycling to the ATC reaction zone 120.

In embodiments, the C2 product stream 426 recovered from the ATC product 126 can comprise ethylene and acetylene. At least a portion of the C2 product stream, a polar aprotic solvent, and hydrogen can be introduced to a liquid phase hydrogenation unit 440, wherein the liquid phase hydrogenation unit comprises an acetylene hydrogenation catalyst, and wherein at least a portion of the acetylene of the C2 product stream can be selectively hydrogenated to produce ethylene.

Nonlimiting examples of polar aprotic solvents suitable for use in the present disclosure include N-methyl-2-pyrrolidone (NMP), N,N-dimethylformamide (DMF), acetone, tetrahydrofuran (THF), dimethylsulfoxide (DMSO), and the like, or combinations thereof.

The liquid phase hydrogenation unit 440 can be any suitable liquid phase hydrogenation reactor, such as a fixed bed catalytic reactor (typically operated adiabatically); and/or a tubular reactor (typically operated isothermally). Generally, the liquid phase hydrogenation unit 440 comprises an acetylene hydrogenation catalyst, such as a palladium (Pd) based catalyst, which can be supported on alumina, zeolites, etc. The hydrogenation catalyst can further comprise other metals, such as platinum, silver, nickel, etc. In embodiments, the acetylene hydrogenation catalyst can comprise Pd/Al₂O₃. Liquid phase hydrogenation of acetylene processes are described in more detail in U.S. Pat. No. 4,128,595, which is incorporated by reference herein in its entirety.

In embodiments, a process for producing olefins can comprise (a) reacting, via an OCM reaction, an OCM reactant mixture in an OCM reaction zone 110 to produce an OCM product 107, wherein the OCM reaction zone 110 comprises an OCM catalyst, wherein the first reactant mixture comprises methane in a methane stream 105 and oxygen in an oxygen stream 106, and wherein the OCM product 107 comprises ethylene, ethane, hydrogen, carbon monoxide, carbon dioxide, and unreacted methane; (b) introducing an ATC feed stream 125 comprising at least a portion of the OCM product 107 and supplemental ethane (e.g., from ethane stream 456 via ethane recycle line 141A) to an ATC reaction zone 120 to produce an ATC product 126, wherein the ATC reaction zone 120 excludes a catalyst, wherein a common reactor III comprises both the OCM reaction zone 110 and the ATC reaction zone 120, wherein a mole ratio of oxygen to ethane in the ATC feed stream 125 is less than or equal to about 0.25, wherein at least a portion of ethane of the ATC feed stream 125 undergoes a cracking reaction to produce ethylene and hydrogen, wherein the ATC product 126 comprises ethylene, ethane, hydrogen, carbon monoxide, carbon dioxide, and unreacted methane, and wherein an amount of ethylene in the ATC product 126 is greater than an amount of ethylene in the OCM product 107; (c) recovering methane 416, ethane 456, propane 466, carbon dioxide 406, ethylene 455, propylene 465, C2+ alkanes (e.g., ethane 456 and propane 466), C3+ alkanes, C4+ hydrocarbons, or a combination thereof from at least a portion of the ATC product 126; and (d) recycling at least a portion of the ethane 456 recovered from the at least a portion of the ATC product 126 as supplemental ethane, respectively.

Herein disclosed are an energy efficient process and system for converting the ethane in an OCM product 107 from an OCM reactor/reaction zone 110 to olefin(s) (e.g., ethylene). In embodiments, the integration of ATC with OCM, as described herein, enables conversion of components (e.g., ethane, propane, and butane) of the OCM product 107 into olefins (e.g., ethylene, propylene) without supplying additional heat (e.g., via a furnace). In conventional technologies, in order to convert the paraffins produced in OCM to olefins, additional steam cracker furnaces are needed. However the system and method of this disclosure, direct conversion of paraffins to olefins can be effected without the use of additional steam cracker furnaces downstream of OCM reactor 110 and upstream of the ATC reaction zone 120. Via the system and method of this disclosure, heat generated by OCM reactions is utilized to make additional olefins via ATC.

In embodiments, herein disclosed is a process for producing ethylene by combining OCM and an ATC reaction zone 120, wherein ethane in the OCM product 107 is converted to ethylene utilizing the high temperature generated by the OCM reactions. An optimal residence time in the (e.g., integrated) pyrolysis reaction zone 120 can be selected, and can be, for example, in a range of from about 200, 250, 300, 400, or 500 ms to about 500, 600, 700, 800, 900, or 1000 milliseconds. Via the system and method provided herein, olefin production can be maximized with a minimum loss of carbon to heavies. As detailed hereinabove, oxygen slip from the OCM can be optimized to a level where the mild oxidation (exothermic) of this oxygen slip provides extra heat for endothermic ATC reactions in the post pyrolysis reaction zone 120. In embodiments, the oxygen slip (e.g., O₂/ethane) can be maintained at less than 0.25, on a molar basis. In embodiments, the OCM reaction zone 110 can be operated at higher pressure than conventional (e.g., conventional OCM operating pressure of less than 1 bar (0.1 MPa)). For example, in embodiments, the OCM is operated at a pressure in a range of from about 3 to about 10 bars (about 0.3 MPa to about 1.0 MPa). A methane conversion provided by the OCM, alone or in combination with the ATC, can be in a range of from about 15 to about 20%.

In embodiments, a process for producing olefins as disclosed herein can advantageously display improvements in one or more process characteristics when compared to an otherwise similar process that does not integrate OCM with ATC for producing desired products. Ethane cracking as disclosed herein can advantageously increase ethylene production.

In embodiments, a process for producing olefins as disclosed herein can advantageously utilize the reaction heat of the OCM reaction for ethane cracking via ATC. Additional advantages of the processes for the production of olefins (e.g., ethylene, propylene) as disclosed herein can be apparent to one of skill in the art viewing this disclosure.

The present disclosure is further illustrated by the following examples, which are not to be construed in any way as imposing limitations upon the scope thereof. On the contrary, it is to be clearly understood that resort can be had to various other aspects, embodiments, modifications, and equivalents thereof which, after reading the description herein, can be suggest to one of ordinary skill in the art without departing from the spirit of the present invention or the scope of the appended claims.

### EXAMPLES

The subject matter having been generally described, the following examples are given as particular embodiments of the disclosure and to demonstrate the practice and advantages thereof. It is understood that the examples are given by way of illustration and are not intended to limit the specification of the claims to follow in any manner.

### Example 1

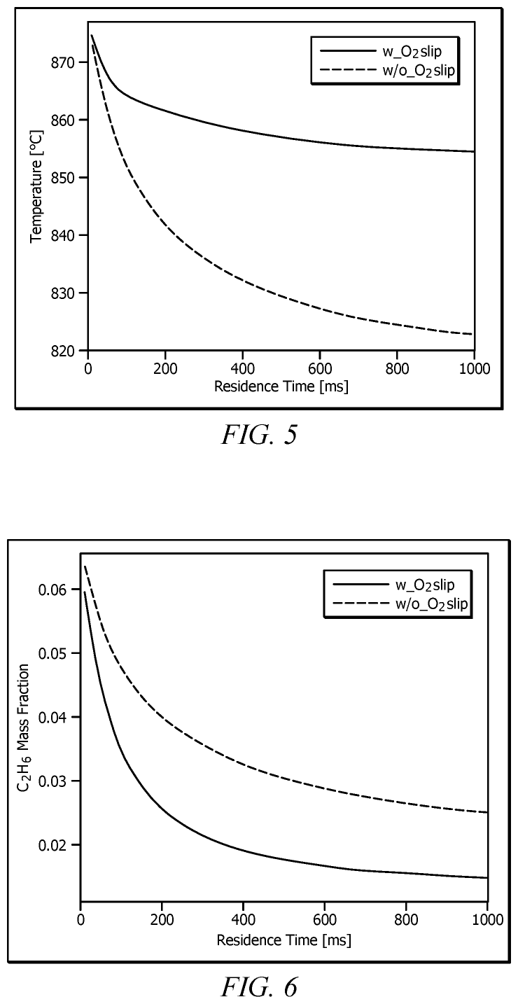

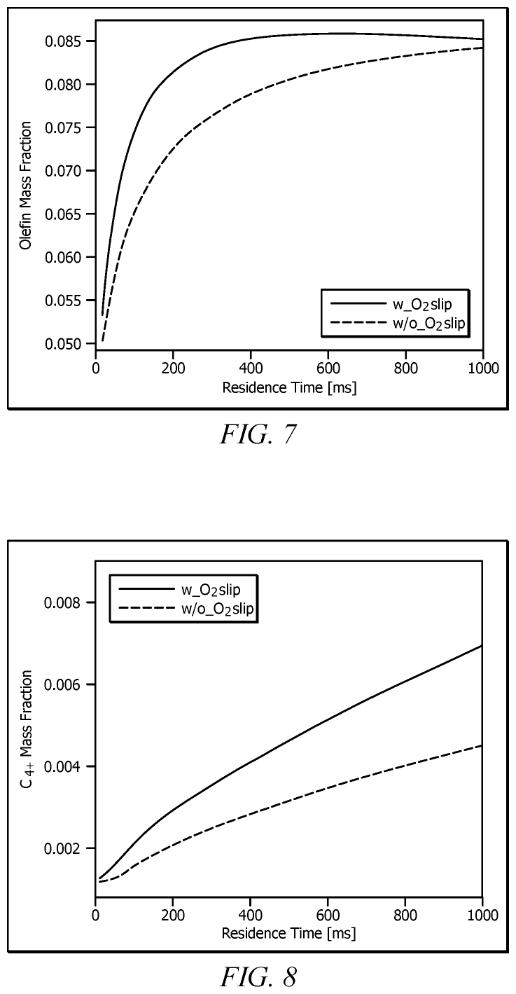

To estimate reactor performance in the pyrolysis reaction zone 120, a detailed kinetic model was simulated using a typical OCM reactor effluent at a pressure of 6 bar (0.6 MPa). FIG. 5 is a schematic of temperature as a function of residence time (tᵣₑₛ) in ATC reaction zone/reactor 120; FIG. 6 is a schematic of ethane mass fraction as a function of residence time (tᵣₑₛ) in ATC reaction zone/reactor 120; FIG. 7 is a schematic of olefin mass fraction as a function of (tᵣₑₛ) in ATC reaction zone/reactor 120; and FIG. 8 is a schematic of C4+ mass fraction as a function of (tᵣₑₛ) in ATC reaction zone/reactor 120. FIGS. 5-8, thus respectively depict temperature, ethane mass fraction, mass fraction of olefins (including, for example, C₂H₂, C₂H₄, C₃H₆), and C4+ (including C4+ paraffins, C4+ olefins, etc.) mass fraction as a function of residence time. Since the operation of the OCM reactor 110 involves oxygen “slip” or passage from the OCM reactor 110 to the ATC reactor 120, the effect of this oxygen slip is also estimated with a 1 mole % of O₂ (e.g., a molar ratio of O₂/ethane of about 0.25).

In the absence of O₂ slip (dashed lines), the rate of olefin production increases rapidly at the entrance of the pyrolysis reactor 120, and levels off at around the residence time of 1000 milliseconds. In contrast, in the presence of O₂ slip (solid lines), the olefin production shows a maximum at around the residence time of 500 milliseconds, and decreases with increasing residence time. Ethane conversion is predicted to be around 71% with O₂ slip and 61% without O₂ slip, respectively. Both cases show continuous increase of heavies (C4+) along the ATC reactor 120. ATC reactor 120 temperature decreases along with increasing residence time due to the temperature reduction by dehydrogenation of hydrocarbons (mainly ethane and propane). A main difference in the temperature profile is that the model predicts a higher temperature profile with O₂ slip than without O₂ slip. This can be mainly due to oxidation reactions incurred by the slipped O₂ along the ATC reactor 120. This mild oxidation provides extra heat, which thermodynamically benefits the overall chemistry (e.g., the endothermic reactions) in the ATC reactor/reaction zone 120.

In this Example, an optimal residence time in the integrated pyrolysis reactor 120 that can maximize the olefin production at the minimum loss of carbon to heavies is around 500 to 1000 milliseconds. The presence of oxygen slip from the OCM reactor/reaction zone 110 benefits the overall performance in the ATC reaction zone 120 by providing extra heat; however, too much oxygen slip (O₂/ethane greater than 0.25, molar basis) can cause a significant temperature increase in the ATC reactor/reaction zone 120, and undesirably oxide all hydrocarbons to COx.

For the purpose of any U.S. national stage filing from this application, all publications and patents mentioned in this disclosure are incorporated herein by reference in their entireties, for the purpose of describing and disclosing the constructs and methodologies described in those publications, which might be used in connection with the methods of this disclosure. Any publications and patents discussed herein are provided solely for their disclosure prior to the filing date of the present application. Nothing herein is to be construed as an admission that the inventors are not entitled to antedate such disclosure by virtue of prior invention.

In any application before the United States Patent and Trademark Office, the Abstract of this application is provided for the purpose of satisfying the requirements of 37 C.F.R. § 1.72 and the purpose stated in 37 C.F.R. § 1.72(b) “to enable the United States Patent and Trademark Office and the public generally to determine quickly from a cursory inspection the nature and gist of the technical disclosure.” Therefore, the Abstract of this application is not intended to be used to construe the scope of the claims or to limit the scope of the subject matter that is disclosed herein. Moreover, any headings that can be employed herein are also not intended to be used to construe the scope of the claims or to limit the scope of the subject matter that is disclosed herein. Any use of the past tense to describe an example otherwise indicated as constructive or prophetic is not intended to reflect that the constructive or prophetic example has actually been carried out.

### Additional Disclosure

In a first embodiment, a method comprises producing an oxidative coupling of methane (OCM) product comprising olefins via oxidative coupling of methane; subjecting an adiabatic thermal cracking (ATC) feed stream comprising at least a portion of the OCM product to ATC (also referred to as “pyrolysis”) to produce an ATC product; and controlling a mole ratio of oxygen to ethane in the ATC feed stream within a range of greater than zero and less than 0.25 (e.g., 0.005 to 0.25, 0.01 to 0.25, 0.05 to 0.25).

A second embodiment can include the method of the first embodiment further comprising utilizing a residence time (tᵣₑₛ), during the subjecting of the ATC feed stream to ATC, that is in a range of from about 200 to about 1000 milliseconds (ms), from about 200 to about 800 ms, or from about 450 to about 550 ms, or less than or equal to about 1000, 900, 800, 700, 600, 550, 500, 450, 400, 350, 300, 250, or 200 milliseconds (ms).

A third embodiment can include the method of the first or the second embodiment further comprising quenching the ATC product immediately after the residence time.

A fourth embodiment can include the method of the third embodiment, wherein quenching further comprises indirectly contacting the ATC product with a heat transfer fluid.

A fifth embodiment can include the method of any one of the first to fourth embodiments, wherein the subjecting the ATC feed stream to ATC to produce the ATC product is effected at a pressure of greater than or equal to about 1, 2, 3, 4, 5, 6, 7, 8, 9, or 10 bars (0.1 MPa, 0.2 MPa, 0.3 MPa, 0.4 MPa, 0.5 MPa, 0.6 MPa, 0.7 MPa, 0.8 MPa, 0.9 MPa, or 1.0 MPa) (e.g., in a range of from about 4 to about 10 bars (from about 0.4 MPa to about 1.0 MPa), from about 4.5 to about 10 bars (from about 0.45 MPa to about 1.0 MPa), or from about 5 to about 6 bars (from about 0.5 MPa to about 0.6 MPa)).

A sixth embodiment can include the method of any one of the first to fifth embodiments, wherein controlling the mole ratio of oxygen to ethane in the ATC feed stream comprises adjusting a mole ratio of oxygen to ethane in the OCM product, adjusting an amount of ethane and/or oxygen that is introduced into the ATC feed stream separately from the at least the portion of the OCM product, or a combination thereof.

A seventh embodiment can include the method of the sixth embodiment, wherein adjusting the mole ratio of the oxygen to ethane in the OCM product further comprises altering a temperature and/or pressure of the ATC during the producing of the OCM product, adjusting an amount of methane and/or oxygen utilized during the producing of the OCM product, or a combination thereof.

An eighth embodiment can include the method of any one of the sixth to seventh embodiments, wherein adjusting the mole ratio of the oxygen to ethane in the OCM product further comprises separating oxygen from or adding oxygen to the at least the portion of the OCM product in the ATC feed stream.

A ninth embodiment can include the method of any one of the sixth to eighth embodiments, wherein adjusting the amount of ethane and/or oxygen introduced into the ATC feed stream separately from the at least the portion of the OCM product further comprises separating ethane from the ATC product and adjusting an amount of the ethane separated from the ATC product that is combined with the at least the portion of the OCM product to provide the ATC feed stream.

A tenth embodiment can include the method of any one of the first to ninth embodiments further comprising separating C2+ alkanes and/or C4+ olefins from the ATC product.

An eleventh embodiment can include the method of the tenth embodiment further comprising increasing a conversion of ethane to olefins by utilizing at least a portion of the C2+ alkanes and/or C4+ olefins separated from the ATC product in the ATC feed stream.

A twelfth embodiment can include the method of the eleventh embodiment, wherein the ethane conversion is greater than or equal to about 40, 45, 50, 55, 60, 65, or 70 mole percent (mol %).

A thirteenth embodiment can include the method of the twelfth embodiment, wherein the ethane conversion is greater than or equal to 70 mol %.

A fourteenth embodiment can include the method of any one of the first to thirteenth embodiments, wherein the OCM is effected at a temperature in a range of from about 860 to 900° C., from about 870 to about 890° C., or from about 875 to about 885° C. (e.g., 880° C.±20° C.).

A fifteenth embodiment can include the method of any one of the first to fourteenth embodiments, wherein the ATC is effected without utilizing a furnace or other heater to increase a temperature of the ATC feed stream.

A sixteenth embodiment can include the method of any one of the first to fifteenth embodiments, wherein the producing the OCM product comprising olefins via OCM and the subjecting the ATC feed stream comprising the at least a portion of the OCM product to ATC to produce the ATC product are effected in a same vessel or in different vessels.

A seventeenth embodiment can include the method of any one of the first to the fifteenth embodiments, wherein the producing the OCM product comprising olefins via OCM is effected in the presence of an OCM catalyst comprising CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, Sr/Mn—Na₂WO₄, or a combination thereof.

In an eighteenth embodiment, a system comprises: an oxidative coupling of methane (OCM) reaction zone configured to produce an OCM product comprising olefins via oxidative coupling of methane; and an adiabatic thermal cracking (ATC) reaction zone configured to produce an ATC product by subjecting a feed stream comprising at least a portion of the OCM product to ATC (also known as, “pyrolysis”), wherein a mole ratio of oxygen to ethane in the ATC feed stream is within a range of greater than zero and less than 0.25 (e.g., 0.005 to 0.25, 0.01 to 0.25, 0.05 to 0.25).

A nineteenth embodiment can include the system of the eighteenth embodiment, wherein the ATC reaction zone provides a residence time (tᵣₑₛ), during the subjecting of the ATC feed stream to ATC, that is in a range of from about 200 to about 1000 milliseconds (ms), from about 300 to about 1000 ms, from about 200 to about 800 ms, or from about 450 to about 550 ms, or less than or equal to about 1000, 900, 800, 700, 600, 550, 500, 450, 400, 350, 300, 250, or 200 milliseconds (ms).

A twentieth embodiment can include the system of the nineteenth embodiment further comprising a quenching zone downstream from the ATC reaction zone, and configured to quench the ATC product immediately after the residence time.

A twenty first embodiment can include the system of the twentieth embodiment, wherein the quenching zone further comprises a heat exchanger configured to reduce a temperature of the ATC product (e.g., to a quenched temperature of less than or equal to about 600, 550, or 500° C.) via indirect contact thereof with a heat transfer fluid.

A twenty second embodiment can include the system of any one of the eighteenth to twenty first embodiments, wherein the ATC reaction zone has an operating pressure of greater than or equal to about 1, 2, 3, 4, 5, 6, 7, 8, 9, or 10 bars (0.1, 0.2, 0.3, 0.4, 0.5, 0.6, 0.7, 0.8, 0.9, or 1.0 MPa) (e.g., in a range of from about 1 to about 10 bars (from about 0.1 MPa to about 1.0 MPa), from about 4.5 to about 6 bars (from about 0.45 MPa to about 0.6 MPa), or from about 5 to about 10 bars (from about 0.5 MPa to about 1.0 MPa)); for example, the ATC reaction zone can have an operating pressure of greater than or equal to about 1, 2, 3, 4, or 5 bars (0.1, 0.2, 0.3, 0.4, or 0.5 MPa) and/or less than or equal to about 10, 9, 8, 7, 6, or 5 bars (1, 0.9, 0.8, 0.7, 0.6, or 0.5 MPa).

A twenty third embodiment can include the system of any one of the eighteenth to twenty second embodiments, wherein the OCM reaction zone can be operated to adjust a molar ratio of oxygen to ethane in the OCM product, and/or further comprising one or more lines to the ATC reaction zone whereby an amount of ethane and/or oxygen can be introduced into the ATC feed stream separately from the at least the portion of the OCM product, or a combination thereof, whereby the mole ratio of oxygen to ethane in the ATC feed stream can be maintained.

A twenty fourth embodiment can include the system of the twenty third embodiment further comprising a separator configured to adjust the mole ratio of the oxygen to ethane in the OCM product by separating oxygen from or adding oxygen to the at least the portion of the OCM product in the ATC feed stream.

A twenty fifth embodiment can include the system of the twenty fourth embodiment, wherein the separator comprises a catalyst within or upstream of the ATC reaction zone.

A twenty sixth embodiment can include the system of any one of the twenty fourth or twenty fifth embodiments further comprising separation apparatus configured to separate ethane from the ATC product, and a recycle line whereby at least a portion of the separated ethane is combined with the at least the portion of the OCM product to provide the ATC feed stream.

A twenty seventh embodiment can include the system of the twenty sixth embodiment, wherein the separation apparatus is further configured to separate C3+ alkanes and/or C4+ olefins from the ATC product.

A twenty eighth embodiment can include the system of any one of the eighteenth to twenty seventh embodiments operable to provide an ethane conversion of greater than or equal to about 40, 45, 50, 55, 60, 65, or 70 mole percent (mol %).

A twenty ninth embodiment can include the system of the twenty eighth embodiment operable to provide an ethane conversion of greater than or equal to 70 mol %.

A thirtieth embodiment can include the system of any one of the eighteenth to twenty ninth embodiments, wherein the OCM reaction zone has an operating temperature in a range of from about 860 to 900° C., from about 870 to about 890° C., or from about 875 to about 885° C. (e.g., 880° C.±20° C.).

A thirty first embodiment can include the system of any one of the eighteenth to thirtieth embodiments further comprising no furnace or other heater configured to increase a temperature of the ATC feed stream between the OCM reaction zone and the ATC reaction zone.

A thirty second embodiment can include the system of any one of the eighteenth to thirty first embodiments, wherein the OCM reaction zone and the ATC reaction zone are in a same vessel or in different vessels.

A thirty third embodiment can include the system of any one of the eighteenth to thirty second embodiments, wherein the OCM reaction zone comprises an OCM catalyst comprising CeO₂, La₂O₃—CeO₂, Ca/CeO₂, Mn/Na₂WO₄, Li₂O, Na₂O, Cs₂O, WO₃, Mn₃O₄, CaO, MgO, SrO, BaO, CaO—MgO, CaO—BaO, Li/MgO, MnO, W₂O₃, SnO₂, Yb₂O₃, Sm₂O₃, MnO—W₂O₃, MnO—W₂O₃—Na₂O, MnO—W₂O₃—Li₂O, SrO/La₂O₃, Ce₂O₃, La/MgO, La₂O₃—CeO₂—Na₂O, La₂O₃—CeO₂—CaO, Na₂O—MnO—WO₃—La₂O₃, La₂O₃—CeO₂—MnO—WO₃—SrO, Na—Mn—La₂O₃/Al₂O₃, Na—Mn—O/SiO₂, Na₂WO₄—Mn/SiO₂, Na₂WO₄—Mn—O/SiO₂, Na/Mn/O, Na₂WO₄, Mn₂O₃/Na₂WO₄, Mn₃O₄/Na₂WO₄, MnWO₄/Na₂WO₄, MnWO₄/Na₂WO₄, Mn/WO₄, Na₂WO₄/Mn, Sr/Mn—Na₂WO₄, or a combination thereof.

In a thirty fourth embodiment, a method comprises: producing an oxidative coupling of methane (OCM) product comprising olefins via oxidative coupling of methane; and subjecting an adiabatic thermal cracking (ATC) feed stream comprising at least a portion of the OCM product to ATC (also known as, “pyrolysis”) to produce an ATC product, wherein a residence time (tᵣₑₛ) of the subjecting of the ATC feed stream to ATC is in a range of from about 200 to about 1000 milliseconds (ms), from about 300 to about 1000 ms, from about 200 to about 800 ms, or from about 450 to about 550 ms, or less than or equal to about 1000, 900, 800, 700, 600, 550, 500, 450, 400, 350, 300, 250, or 200 milliseconds (ms).

A thirty fifth embodiment can include the method of the thirty fourth embodiment further comprising controlling a mole ratio of oxygen to ethane in the ATC feed stream within a range of greater than zero and less than 0.25 (e.g., 0.005 to 0.25, 0.01 to 0.25, 0.05 to 0.25, 0.005 to 0.2, 0.01 to 0.2, 0.05 to 0.2).

While embodiments of the disclosure have been shown and described, modifications thereof can be made without departing from the spirit and teachings of the invention. The embodiments and examples described herein are exemplary only, and are not intended to be limiting. Many variations and modifications of the invention disclosed herein are possible and are within the scope of the invention.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the detailed description of the present invention. The disclosures of all patents, patent applications, and publications cited herein are hereby incorporated by reference.

1. A method comprising:
producing an oxidative coupling of methane (OCM) product comprising olefins via oxidative coupling of methane;
subjecting an adiabatic thermal cracking (ATC) feed stream comprising at least a portion of the OCM product to ATC to produce an ATC product; and
controlling a mole ratio of oxygen to ethane in the ATC feed stream within a range of greater than zero and less than 0.25.

2. The method of claim 1 further comprising utilizing a residence time (tᵣₑₛ), during the subjecting of the ATC feed stream to ATC, that is in a range of from about 200 to about 1000 milliseconds (ms).

3. The method of claim 2 further comprising quenching the ATC product immediately after the residence time.

4. The method of claim 1, wherein controlling the mole ratio of oxygen to ethane in the ATC feed stream comprises adjusting a mole ratio of oxygen to ethane in the OCM product, adjusting an amount of ethane and/or oxygen that is introduced into the ATC feed stream separately from the at least the portion of the OCM product, or a combination thereof.

5. The method of claim 4:
wherein adjusting the mole ratio of the oxygen to ethane in the OCM product further comprises altering a temperature, a pressure, a temperature of an OCM feed, a flowrate of the OCM feed, and/or a catalyst loading in the OCM zone during the producing of the OCM product, adjusting an amount of methane and/or oxygen utilized during the producing of the OCM product, or a combination thereof, or
wherein adjusting the mole ratio of the oxygen to ethane in the OCM product further comprises separating oxygen from or adding oxygen to the at least the portion of the OCM product in the ATC feed stream, or wherein adjusting the amount of ethane and/or oxygen introduced into the ATC feed stream separately from the at least the portion of the OCM product further comprises separating ethane from the ATC product and adjusting an amount of the ethane separated from the ATC product that is combined with the at least the portion of the OCM product to provide the ATC feed stream.

6. The method of claim 1, wherein an ethane conversion in ATC is greater than or equal to about 70 mole percent (mol %), or wherein the OCM is effected at a temperature in a range of from about 860 to 900° C.

7. A system comprising:
an oxidative coupling of methane (OCM) reaction zone configured to produce an OCM product comprising olefins via oxidative coupling of methane; and
an adiabatic thermal cracking (ATC) reaction zone configured to produce an ATC product by subjecting a feed stream comprising at least a portion of the OCM product to ATC, wherein a mole ratio of oxygen to ethane in the ATC feed stream is within a range of greater than zero and less than 0.25.

8. The system of claim 7, wherein the ATC reaction zone provides a residence time (tᵣₑₛ), during the subjecting of the ATC feed stream to ATC, that is in a range of from about 200 to about 1000 milliseconds (ms).

9. The system of claim 8 further comprising a quenching zone downstream from the ATC reaction zone, and configured to quench the ATC product immediately after the residence time.

10. The system of claim 7, wherein the ATC reaction zone has an operating pressure of greater than or equal to about 1 bars (0.1 MPa) and less than 10 bars (1 MPa), or wherein the OCM reaction zone can be operated to adjust a molar ratio of oxygen to ethane in the OCM product, and/or further comprising one or more lines to the ATC reaction zone whereby an amount of ethane and/or oxygen can be introduced into the ATC feed stream separately from the at least the portion of the OCM product, or a combination thereof, whereby the mole ratio of oxygen to ethane in the ATC feed stream can be maintained.

11. The system of claim 10 further comprising a separator configured to adjust the mole ratio of the oxygen to ethane in the OCM product by separating oxygen from or adding oxygen to the at least the portion of the OCM product in the ATC feed stream.

12. The system of claim 11 further comprising separation apparatus configured to separate ethane from the ATC product, and a recycle line whereby at least a portion of the separated ethane is combined with the at least the portion of the OCM product to provide the ATC feed stream.

13. The system of claim 7, wherein the system is operable to provide an ethane conversion of greater than or equal to about 70 mole percent (mol %), or wherein the OCM reaction zone has an operating temperature in a range of from about 860 to 900° C.

14. A method comprising:
producing an oxidative coupling of methane (OCM) product comprising olefins via oxidative coupling of methane; and
subjecting an adiabatic thermal cracking (ATC) feed stream comprising at least a portion of the OCM product to ATC to produce an ATC product, wherein a residence time (tᵣₑₛ) of the subjecting of the ATC feed stream to ATC is in a range of from about 200 to about 1000 milliseconds (ms) milliseconds (ms).

15. The method of claim 14 further comprising controlling a mole ratio of oxygen to ethane in the ATC feed stream within a range of greater than zero and less than 0.25.
